SEQUENCE LISTING (2 sequences)

SEQ ID NO: 1 - DNA, 846 bp, Homo sapiens
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 2 - DNA, 780 bp, Homo sapiens
atggcccggatccccaagaccctaaagttcgtcgtcgtcatcgtcgcggtcctgctgccagtcctagcttactctgccaccactgcccggcaggaggaagttccccagcagacagtggccccacagcaacagaggcacagcttcaagggggaggagtgtccagcaggatctcatagatcagaacatactggagcctgtaacccgtgcacagagggtgtggattacaccaacgcttccaacaatgaaccttcttgcttcccatgtacagtttgtaaatcagatcaaaaacataaaagttcctgcaccatgaccagagacacagtgtgtcagtgtaaagaaggcaccttccggaatgaaaactccccagagatgtgccggaagtgtagcaggtgccctagtggggaagtccaagtcagtaattgtacgtcctgggatgatatccagtgtgttgaagaatttggtgccaatgccactgtggaaaccccagctgctgaagagacaatgaacaccagcccggggactcctgccccagctgctgaagagacaatgaacaccagcccggggactcctgccccagctgctgaagagacaatgaccaccagcccggggactcctgccccagctgctgaagagacaatgaccaccagcccggggactcctgccccagctgctgaagagacaatgatcaccagcccggggactcctgcctcttctcattacctctcatgcaccatcgtagggatcatagttctaattgtgcttctgattgtgtttgtttga

(19) United States
(12) Patent Application Publication
(10) Pub. No.: US 2021/0338738 A1
(43) Pub. Date: Nov. 4, 2021

(54) CELLS ENGINEERED FOR CO-EXPRESSION OF DECOY RECEPTOR 1 AND TNF-RELATED APOPTOSIS-INDUCING LIGAND AND USES THEREFOR

(71) Applicant: Vanderbilt University, Nashville, TN (US)

(72) Inventors: Michael R. KING, Nashville, TN (US); Nidhi JYOTSANA, Nashville, TN (US)

(73) Assignee: Vanderbilt University, Nashville, TN (US)

(21) Appl. No.: 17/306,153

(22) Filed: May 3, 2021

Related U.S. Application Data
(60) Provisional application No. 63/018,964, filed on May 1, 2020.

(51) Int. Cl. A61K 35/28; C12N 5/0789; A61P 35/00; A61K 9/00; A61K 45/06; C07K 14/705

(52) U.S. Cl. CPC A61K 35/28; C12N 5/0647; A61P 35/00; A61K 9/0019; C07K 2319/50; A61K 45/06; C07K 14/70575; C07K 14/70578; C12N 2510/00; A61K 9/0014

(57) ABSTRACT

The present disclosure describes engineering of cells to co-express TNF-Related Apoptosis-Inducing Ligand (TRAIL) and Decoy Receptor 1 (DcR1). The expression of DcR1 results in competition for TRAIL binding to Death Receptors 4 and 5, thereby protecting the engineered cells from TRAIL-induced apoptosis. Such cells will exhibit longer survival such as when used in cell-based therapies for cancer.

### PRIORITY CLAIM

This application claims benefit of priority to U.S. Provisional Application Ser. No. 63/018,964, filed May 1, 2020, the entire contents of which are hereby incorporated by reference

### FEDERAL FUNDING SUPPORT CLAUSE

This invention was made with government support under grant number R01 CA203991 awarded by the National Institutes of Health. The government has certain rights in the invention.

### BACKGROUND

### 1. Field

The present invention relates generally to the fields of molecular biology, oncology, and medicine. More particularly, it concerns engineering of cells to co-express TNF-Related Apoptosis-Inducing Ligand (TRAIL) and Decoy Receptor 1 (DcR1).

### 2. Description of Related Art

Tumor necrosis factor-related apoptosis-inducing ligand (TRAIL) has been used to target different types of cancer cells in pre-clinical and clinical settings with no major side effects. TRAIL binds to death receptors 4 and 5 (DR4 and DR5) and induces caspase3/8 dependent apoptosis in cancer cells. However, very short half-life of TRAIL recombinant protein (approx. 30 minutes) in vivo presents a major challenge in its clinical application. To avoid this delivery problem and to establish an endogenous TRAIL expressing machinery in vivo, genetically engineering of cells to stably express higher amounts of TRAIL protein is a goal.

Unfortunately, engineered cells that express TRAIL have a transient therapeutic window following transplantation. This has been determined to be at least partly due to TRAIL overexpression, which result in an increase in apoptosis in the engineered cells both, in vitro and in vivo, results. This leads to a decrease in the engraftment of TRAIL-expressing cells. Thus, improvements in cell-based TRAIL therapy that produce both robust and long-lasting therapeutic efficacy are needed.

### SUMMARY

In one embodiment, the present disclosure provides for an engineered cell that expresses Decoy Receptor 1 (DcR1) and TNF-Related Apoptosis-Inducing Ligand (TRAIL) and exhibits reduced activation of caspase-dependent apoptosis as compared a cell engineered to express TRAIL but not engineered to express DcR1. The engineered cell may overexpress TRAIL as compared to a non-engineered cell, may overexpress DcR1 as compared to a non-engineered cell, or may overexpress TRAIL and DcR1 as compared to a non-engineered cell. The engineered cell may be a stem cell, such as a hematopoietic stem cell (HSC). The coding regions for DcR1 and TRAIL are under control of the same promoter, may be separated by an internal ribosome entry site and/or may be encoded as a polyprotein having a protease cleavage site disposed between DcR1 and TRAIL sequences. Alternatively, the coding regions for DcR1 and TRAIL are under control of different promoters. The engineered cell may be a non-human mammalian cell, such as a murine cell, or a human cell. The engineered cell may further express a detectable marker protein, and/or may encode an inducible suicide gene.

In another embodiment, there is provided a method of killing a cancer cell comprising contacting said cancer cell with an engineered cell as described herein. The cancer cell may be is a breast cancer cell (including s triple negative breast cancer cell), a lung cancer cell, a skin cancer cell, a brain cancer cell, a head & neck, a lymphatic cancer cell, a pancreatic cancer cell, a liver cancer cell, a bladder cancer cell, a stomach cancer cell, a colon cancer cell, a prostate cancer cell, a uterine cancer cell, a cervical cancer cells, a testicular cancer cell, a lymphoma cell, a leukemia cell. The cancer cell may be a recurrent, metastatic or drug resistant cancer cell.

Contacting may comprise intra-tumoral administration, administration into the tumor vasculature, or administration regional to the cancer, or may comprise systemic administration, such as intravascular (intravenous, intra-arterial), intraperitoneal, subcutaneous or topical administration. The method may further comprise contacting said cancer cell with a second cancer therapy, such as chemotherapy, radiotherapy, immunotherapy, hormonal therapy, toxin therapy. The cancer cell may be located in a living subject and the method may further comprise a conditioning treatment to improve engraftment of said engineered cell. The method may further comprise performing surgery on said living subject to resect said cancer cell. The living subject may be a human or non-human mammal.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Cellular TRAIL delivery for TNBC therapy. Bone marrow is the factory of blood production and hematopoietic stem cells are the cells that give rise to different types of blood cells. Genetic engineering or gene therapy involving manipulation of HSCs have clearly shown pre-clinical and clinical success for more than 3 decades, opening up new avenues for the treatment of various diseases including cancer. In light of this, the current invention aims to overexpress TRAIL in hematopoietic stem cells for providing an endogenous machinery to fight triple negative breast cancer. It is expected that this stabilized cellular delivery of TRAIL will improve the following by targeting:
1. Primary tumor in 4T1 triple negative breast cancer model;
2. Distant metastasis to lung, liver, lymph node, bone marrow for longer term application;
3. Dormant cancer stem cells that hide in the bone marrow and cause disease relapse

FIG. 2—Full-length TRAIL cloned in MSCV plasmid. Following the hematopoietic stem cell genetic manipulation and transplantation schematics here, full-length human trail in a GFP expressing retroviral MSCV vector was cloned and sequenced.

FIG. 3—Establishing a TRAIL producer cell line. This vector was used to transduce and sort the virus producer cell line that stably expresses TRAIL retrovirus as can be seen that all these cells express GFP. TRAIL expression was confirmed by performing TRAIL ELISA. A 110-fold increase was observed in the levels of TRAIL in the supernatant of these cells.

FIG. 4—Co-culture of TRAIL producer cells with 4T1 cells. When TRAIL producer cell line and 4T1 breast cancer cells were co-cultured in a 50:50 ratio, a decrease in the percentage of 4T1s was observed when cultured with TRAIL producer cells compared to empty vector.

FIG. 5—Hematopoietic stem cells (HSCs) engineered for TRAIL production. Next step was to transduce the hematopoietic stem cells for characterization and in vivo transplantation. So, the bone marrow cells were isolated from a healthy donor mouse and depleted these cells for lineage to obtain the stem and progenitor population. The retroviral transduction of HSCs results in 60 to 75% of GFP positive HSCs in a reproducible manner.

FIG. 6—Engineered HSCs produce TRAIL. Engineered HSCs for TRAIL production were further characterized and found to have a 25-fold increase in TRAIL in the supernatant of these cells.

FIG. 7—HSCs for cancer therapy. For the next step of transplant into the mice, there were two choices: 1st transplantation of engineered hematopoietic stem cells and then inoculation of 4T1 tumor cells or inoculation of 4T1 tumor cells followed by HSC transplantation.

FIG. 8—TRAIL-HSC transplantation followed by tumor inoculation. Transplantation was performed first and lethally irradiated and transplanted the recipient mice with engineered HSCs t that produce TRAIL and GFP followed by 4T1 injections.

FIG. 9—TRAIL-engineered donor HSCs decrease over time. Blood was drawn from these mice via tail vein bleeding at different time points to monitor the engraftment of donor cells in the peripheral blood. The bleeding analysis of these mice suggested almost similar engraftment of empty vector and TRAIL HSCs at two weeks, however the % of trail expressing cells went down with time.

FIG. 10—Short window of therapeutic benefit for TRAIL-HSCs. 4T1 TNBC cells were injected in these mice at day 16 and confirmed the engraftment of donor cells at day 15 in previous slide. and imaged them for luciferase signal of cancer cells at day 16. A decrease in luciferase signal was observed in the TRAIL group from day 25 to day 35. Though a decrease in the signal and a visible necrotic core at primary tumor site was observed, this was only a transient therapeutic effect, mostly due to a decrease in the percentage of TRAIL expressing cells.

FIG. 11—Tumor inoculation followed by TRAIL-HSC transplantation. In the 2nd in vivo experiment, 4T1 tumor cells were first injected on both sides of 4h mammary fat pad, followed by HSC transplantation.

FIG. 12—Tumor inoculation followed by TRAIL-HSC transplantation—GFP expression. This graph shows the percentage of GFP positive cells in peripheral blood of the transplanted mice and again, a proliferative disadvantage of TRAIL expressing HSCs is observed. Again, a therapeutic benefit was observed between day 2 and day 10 and between day 18 and day 30. And in these mice, a visible necrotic core was observed in the tumor. Again, this effect was transient as these mice eventually died or had to be sacrificed because of tumor size and metastatic burden.

FIG. 13—Tumor inoculation followed by TRAIL-HSC transplantation—survival. A trend of increased survival was observed in the TRAIL-HSCs transplanted group. This suggests that there is a potential anti-cancer therapeutic effect of the transplanted TRAIL-expressing HSCs.

FIG. 14—TRAIL expression may induce apoptosis in HSCs. In all of the in vivo experiments, it was clearly observed that TRAIL expressing HSCs do not have proliferative advantage in vivo and that they decrease in number with time. It was hypothesized that the overexpression of TRAIL in these cells may lead to their apoptosis. So, to confirm this, the percentage of apoptotic cells in TRAIL expressing HSCs was quantified both in vitro and in vivo and showed an increased apoptosis in HSCs that overexpress TRAIL.

FIG. 15—Decoy receptor 1 expression can avoid apoptosis in cells. The data here show that the K562 leukemia cells overexpress DcR1 following transduction with DcR1-retroviral vector and as it was expected that they could be rescued from TRAIL-mediated apoptosis significantly.

FIG. 16—Nucleotide sequences for TRAIL and DcR1 used for cloning.

FIG. 17—Retroviral MSCV-IRES-GFP vector map.

FIG. 18—pLV-eGFP plasmid map.

FIG. 19—Co-expression of DcR1 and TRAIL decreases TRAIL mediated apoptosis in transduced hematopoietic stem cells (HSCs) in vitro. Isolated mouse bone marrow HSCs were transduced with 1) EV: empty GFP vector, 2) FLT: full length TRAIL, 3) DcR1: Dcoy receptor 1 and empty GFP vector, 4) FLT-DcR1: full length TRAIL plus Decoy receptor 1. The percentage of viable cells was measured via Annexin V-PI assay following 72 h of retroviral transduction in vitro.

FIGS. 20A-C—Transplantation of either TRAIL or DcR1 and TRAIL co-expressing HSCs in decreases tumor burden in 4T1-TNBC mouse model. Isolated mouse bone marrow HSCs were transduced with 1) EV: empty GFP vector, 2) FLT: full length TRAIL, 3) DcR1: Dcoy receptor 1 and empty GFP vector, 4) FLT-DcR1: full length TRAIL plus Decoy receptor 1. (FIG. 20A) In vivo luminescence imaging of 4T1-mice at different time points following transplantation with retrovirally transduced HSCs. (FIG. 20B) Quantitation of tumor burden following in vivo luminescence imaging. (FIG. 20C) Tumor volume at day 27 following 4T1 injection in mammary fat pad of mice.

### DESCRIPTION OF ILLUSTRATIVE EMBODIMENTS

Decoy receptor 1 (DcR1) is a TRAIL receptor that competes with DR4 and DR5 for TRAIL binding. The inventors and others have observed that DcR1-TRAIL binding does not lead to the activation of caspase-dependent apoptotic pathway and thus reduces/prevents the killing of cells that overexpress DcR1. In order to facilitate a prolonged engraftment/enhanced anti-cancer effect and to avoid apoptosis in TRAIL expressing cells, the inventors co-expressed DcR1 and TRAIL in hematopoietic stem cells (HSCs). The results in K562 leukemia cells confirm that overexpression of DcR1 prevents them from TRAIL-mediated apoptosis in a significant manner. The inventors thereby propose that over expression of DcR1 in TRAIL expressing HSCs will result in a prolonged engraftment when introduced into a cancer patient which in term leads to enhanced anti-cancer effects. These and other aspects of the disclosure are set out in detail below.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “about” means in general, the stated value plus or minus 5%.

“Treatment” and “treating” refer to administration or application of a therapeutic agent to a subject or performance of a procedure or modality on a subject for the purpose of obtaining a therapeutic benefit of a disease or health-related condition.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

As used herein, a “stem cell” is a cell with the developmental potential to produce a more specialized cell type and at the same time to replicate itself. A stem cell may divide to produce two daughters that are themselves stem cells or it may divide to produce a daughter that is a stem cell and a daughter that is a more specialized cell type. A stem cell may originate from the embryo, fetus, or adult.

As used herein, a “hematopoietic stem cell” is a stem cell with the developmental potential to produce any type of blood cell, such as white blood cells, red blood cells, and platelets.

A “somatic cell” is defined herein as a diploid cell of any tissue type that does not contribute to the propagation of the genome beyond the current generation of the organism. All cells except for germ cells are somatic cells and constitute the individual's body.

The terms “cell culture” and “culture” encompass the maintenance of cells in an artificial, in vitro environment. It is to be understood, however, that the term “cell culture” is a generic term and may be used to encompass the cultivation not only of individual cells, but also of tissues, organs, organ systems or whole organisms, for which the term “tissue culture” may occasionally be used interchangeably with the term “cell culture.”

The terms “cell culture medium” or “culture medium” (plural “media” in each case) refer to a nutritive solution for cultivating cells and may be used interchangeably.

### II. TUMOR NECROSIS FACTOR-RELATED APOPTOSIS-INDUCING LIGAND (TRAIL)

TNF-related apoptosis-inducing ligand (TRAIL) is a protein functioning as a ligand that induces the process of cell death called apoptosis. TRAIL is a cytokine that is produced and secreted by most normal tissue cells. It causes apoptosis primarily in tumor cells, by binding to certain death receptors. TRAIL and its receptors have been used as the targets of several anti-cancer therapeutics since the mid-1990s, such as Mapatumumab. However, as of 2013, these have not shown significant survival benefit. TRAIL has also been implicated as a pathogenic or protective factor in various pulmonary diseases, particularly pulmonary arterial hypertension.

TRAIL has also been designated CD253 (cluster of differentiation 253) and TNFSF10 (tumor necrosis factor (ligand) superfamily, member 10). In humans, the gene that encodes TRAIL is located at chromosome 3q26, which is not close to other TNF family members. The genomic structure of the TRAIL gene spans approximately 20 kb and is composed of five exonic segments 222, 138, 42, 106, and 1245 nucleotides and four introns of approximately 8.2, 3.2, 2.3 and 2.3 kb.

The TRAIL gene lacks TATA and CAAT boxes and the promoter region contains putative response elements for transcription factors GATA, AP-1, C/EBP, SP-1, OCT-1, AP3, PEAS, CF-1, and ISRE. TIC10 (which causes expression of TRAIL) was investigated in mice with various tumor types. The small molecule ONC201 causes expression of TRAIL which kills some cancer cells.

TRAIL shows homology to other members of the tumor necrosis factor superfamily. It is composed of 281 amino acids and has characteristics of a type II transmembrane protein. The N-terminal cytoplasmic domain is not conserved across family members; however, the C-terminal extracellular domain is conserved and can be proteolytically cleaved from the cell surface. TRAIL forms a homotrimer that binds three receptor molecules.

TRAIL binds to the death receptors DR4 (TRAIL-RI) and DR5 (TRAIL-RII). The process of apoptosis is caspase-8-dependent. Caspase-8 activates downstream effector caspases including procaspase-3, -6, and -7, leading to activation of specific kinases. TRAIL also binds the receptors DcR1 and DcR2, which do not contain a cytoplasmic domain (DcR1) or contain a truncated death domain (DcR2). DcR1 functions as a TRAIL-neutralizing decoy-receptor. The cytoplasmic domain of DcR2 is functional and activates NFKB. In cells expressing DcR2, TRAIL binding therefore activates NFκB, leading to transcription of genes known to antagonize the death signaling pathway and/or to promote inflammation. Application of engineered ligands that have variable affinity for different death (DR4 and DR5) and decoy receptors (DCR1 and DCR2) may allow selective targeting of cancer cells by controlling activation of Type 1/Type 2 pathways of cell death and single cell fluctuations.

Exemplary protein and nucleic acid sequences for TRAIL are found at NP_033451 and NM_009425, respectively.

### III. DECOY RECEPTOR 1

Decoy receptor 1 (DcR1), also known as TRAIL receptor 3 (TRAILR3) and tumor necrosis factor receptor superfamily member 10C (TNFRSF10C), is a human cell surface receptor of the TNF-receptor superfamily. The protein encoded by this gene is a member of the TNF-receptor superfamily. This receptor contains an extracellular TRAIL-binding domain and a transmembrane domain, but no cytoplasmic death domain. This receptor is not capable of inducing apoptosis and is thought to function as an antagonistic receptor that protects cells from TRAIL-induced apoptosis. This gene was found to be a p53-regulated DNA damage-inducible gene. The expression of this gene was detected in many normal tissues but not in most cancer cell lines, which may explain the specific sensitivity of cancer cells to the apoptosis-inducing activity of TRAIL.

Exemplary protein and nucleic acid sequences for DcR1 are found at NP_003832 and NM_003841, respectively.

### IV. HEMATOPOIETIC STEM CELLS

Hematopoietic stem cells (HSCs) are the stem cells that give rise to other blood cells. This process is called hematopoiesis. This process occurs in the red bone marrow, in the core of most bones. In embryonic development, the red bone marrow is derived from the layer of the embryo called the mesoderm.

Hematopoiesis is the process by which all mature blood cells are produced. It must balance enormous production needs (the average person produces more than 500 billion blood cells every day) with the need to regulate the number of each blood cell type in the circulation. In vertebrates, the vast majority of hematopoiesis occurs in the bone marrow and is derived from a limited number of hematopoietic stem cells that are multipotent and capable of extensive self-renewal.

Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, and natural killer cells. The definition of hematopoietic stem cell has evolved since they were first discovered in 1961. The hematopoietic tissue contains cells with long-term and short-term regeneration capacities and committed multipotent, oligopotent, and unipotent progenitors. Hematopoietic stem cells constitute 1:10,000 of cells in myeloid tissue. They are round, non-adherent, with a rounded nucleus and low cytoplasm-to-nucleus ratio. In shape, hematopoietic stem cells resemble lymphocytes.

Hematopoietic stem cells are found in the bone marrow of adults, especially in the pelvis, femur, and sternum. They are also found in umbilical cord blood and, in small numbers, in peripheral blood. Stem and progenitor cells can be taken from the pelvis, at the iliac crest, using a needle and syringe. The cells can be removed as liquid (to perform a smear to look at the cell morphology) or they can be removed via a core biopsy (to maintain the architecture or relationship of the cells to each other and to the bone).

A colony-forming unit is a subtype of HSC. This sense of the term is different from colony-forming units of microbes, which is a cell counting unit. There are various kinds of HSC colony-forming units:

Colony-forming unit—granulocyte-erythrocyte-monocyte-megakaryocyte (CFU-GEMM)

Colony-forming unit—lymphocyte (CFU-L)

Colony-forming unit—erythrocyte (CFU-E)

Colony-forming unit—granulocyte-macrophage (CFU-GM)

Colony-forming unit—megakaryocyte (CFU-Meg)

Colony-forming unit—basophil (CFU-B)

Colony-forming unit—eosinophil (CFU-Eos)

The above CFUs are based on the lineage. Another CFU, the colony-forming unit—spleen (CFU-S), was the basis of an in vivo clonal colony formation, which depends on the ability of infused bone marrow cells to give rise to clones of maturing hematopoietic cells in the spleens of irradiated mice after 8 to 12 days. It was used extensively in early studies but is now considered to measure more mature progenitor or transit-amplifying cells rather than stem cells.

Since Hematopoietic stem cells cannot be isolated as a pure population, it is not possible to identify them in a microscope. Hematopoietic stem cells can be identified or isolated by the use of flow cytometry where the combination of several different cell surface markers (particularly CD34) are used to separate the rare Hematopoietic stem cells from the surrounding blood cells. Hematopoietic stem cells lack expression of mature blood cell markers and are thus, called Lin-. Lack of expression of lineage markers is used in combination with detection of several positive cell-surface markers to isolate Hematopoietic stem cells. In addition, Hematopoietic stem cells are characterized by their small size and low staining with vital dyes such as rhodamine 123 (rhodamine^{lo}) or Hoechst 33342 (side population).

Hematopoietic stem cell transplantation (HSCT) is the transplantation of multipotent hematopoietic stem cells, usually derived from bone marrow, peripheral blood, or umbilical cord blood. It may be autologous (the patient's own stem cells are used), allogeneic (the stem cells come from a donor) or syngeneic (from an identical twin).

It is most often performed for patients with certain cancers of the blood or bone marrow, such as multiple myeloma or leukemia. In these cases, the recipient's immune system is usually destroyed with radiation or chemotherapy before the transplantation. Infection and graft-versus-host disease are major complications of allogeneic HSCT.

In order to harvest stem cells from the circulating peripheral blood, blood donors are injected with a cytokine, such as granulocyte-colony stimulating factor (G-CSF), that induces cells to leave the bone marrow and circulate in the blood vessels. In mammalian embryology, the first definitive Hematopoietic stem cells are detected in the AGM (aorta-gonad-mesonephros), and then massively expanded in the fetal liver prior to colonizing the bone marrow before birth.

A cobblestone area-forming cell (CAFC) assay is a cell culture-based empirical assay. When plated onto a confluent culture of stromal feeder layer, a fraction of Hematopoietic stem cells creep between the gaps (even though the stromal cells are touching each other) and eventually settle between the stromal cells and the substratum (here the dish surface) or trapped in the cellular processes between the stromal cells. Emperipolesis is the in vivo phenomenon in which one cell is completely engulfed into another (e.g., thymocytes into thymic nurse cells); on the other hand, when in vitro, lymphoid lineage cells creep beneath nurse-like cells, the process is called pseudoemperipolesis, or more commonly CAFC, which means areas or clusters of cells look dull cobblestone-like under phase contrast microscopy, compared to the other Hematopoietic stem cells, which are refractile. This happens because the cells that are floating loosely on top of the stromal cells are spherical and thus refractile. However, the cells that creep beneath the stromal cells are flattened and, thus, not refractile. The mechanism of pseudoemperipolesis is only recently coming to light. It may be mediated by interaction through CXCR4 (CD184) the receptor for CXC Chemokines (e.g., SDF1) and α4β1 integrins.

B. Culture

The method may comprise culturing a population of HSCs. For use in the present disclosure, cells can be plated directly onto the surface of culture vessels without attachment factors. The optimal plating and culture conditions can easily be determined by one of ordinary skill in the art using only routine experimentation.

In some aspects, genetically engineered cells are used, wherein at least one cell of the HSCs is transfected with an exogenous polynucleotide encoding TRAIL, and also transfected with an exogenous polynucleotide encoding Decoy Receptor 1.

Cells are typically cultivated in a cell incubator at about 37° C. The incubator atmosphere is humidified and contains about 3-10% carbon dioxide in air, although cultivation of certain cell lines may require as much as 20% carbon dioxide in air for optimal results. Culture medium pH is in the range of about 7.1-7.6, about 7.1-7.4, or about 7.1-7.3. Cells in closed or batch culture typically undergo complete medium exchange (i.e., replacing spent media with fresh media) every few days as required by the specific cell type, typically about every 2-3 days. Cells in perfusion culture (e.g., in bioreactors or fermenters) receive fresh media on a continuously recirculating basis.

Culture and differentiation agents useful in the present disclosure include, by way of example, the following: medium refers to culture media for cells, as for example DMEM/F12 (Dulbecco's modified Eagle's medium/Ham's F12, 1:1, Invitrogen, Carlsbad, Calif.), also encompassing possible alternatives, variations and improvements equivalent to this cell culture medium. In accordance with the particular needs of the cultured cell, the medium may be supplemented with serum preferably at least 5% serum, and more preferably about 15% serum. According to a particular embodiment of the invention, said serum is from bovine origin, more particularly bovine fetal serum, although synthetic and non-synthetic serums, from human and other animals may also be employed, as well as other synthetic or natural reagents, including mixtures thereof, that allow the culture of the cells.

In some aspects, the medium is serum free medium. The cell culture medium may contain antibiotics such as penicillin and streptomycin and/or amino acids such as glutamine and other non-essential amino acids and mixtures thereof. The cells as described herein may be cultured in the presence of a single agent or multiple agents, concurrently or sequentially, for a variable duration of time. The choice of a specific medium depends on the type of cultured cell and is well within the knowledge of a person skilled in the art.

The term “defined” or “fully defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco' s Modified Eagle's Medium (DMEM), F12, or Roswell Park Memorial Institute Medium (RPMI) 1640, containing amino acids, vitamins, inorganic salts, buffers, antioxidants and energy sources) which is supplemented with recombinant albumin, chemically defined lipids, and recombinant insulin. An exemplary fully defined medium is Essential 8™ medium.

This medium according to the present disclosure may comprise a) base medium, b) supplements, and c) growth factors. The base medium may include commonly used formulations well known to those skilled in the art including: RPMI, other commonly used basal media and preferably MEM or more preferably the alpha modification of MEM (α-MEM). These base media also contain commonly used buffers to maintain physiological pH during the cell culture process, including but not limited to, sodium bicarbonate, HEPES and other buffer substances with a pKa in the physiological pH range. Supplements added to the base medium also include those commonly used in cell culture including transferrin or other iron-chelating agents, insulin (including natural or recombinant forms, insulin-like growth factors I & II, and related substances), trace elements, sodium pyruvate, non-essential amino acids, dextran at various molecular sizes, hydrocortisone, ethanolamine, glucose and the tri-peptide, glycyl-histidine-lysine. The appropriate concentrations & compositions for such supplements will be readily apparent to those skilled in the art. Optimal levels of cell culture medium constituents are often determined through an empirical process of testing potential concentrations against a defined endpoint including for example, the growth rate of the cells, etc. The exact formulation of various basal medium supplements may be varied from the list of specific supplements described above while still retaining the specific characteristics of the present invention that primarily includes the ability to support growth of the mesenchymal cell culture. The concentrations and other ingredients in a formulation of standard cell culture medium are well known to those of ordinary skill in the art.

The present disclosure also contemplates the use of “defined culture media” or “serum-free media” (SFM). A number of SFM formulations are commercially available, such as those designed to support the culture of endothelial cells, keratinocytes, monocytes/macrophages, fibroblasts, chondrocytes, or hepatocytes, which are available from GIBCO/LTI (Gaithersburg, Md.). For example, SFM formulations supporting in vitro culture of keratinocytes have been reported (e.g., U.S. Pat. Nos. 4,673,649 and 4,940,666). The culture media of the present disclosure are typically sterilized to prevent unwanted contamination. The media compositions and formulations of the present disclosure include components which are known to the skilled artisan or can be otherwise deduced using routine methods.

In some aspects, the cultured cells may be reinforced with exogenously added extracellular matrix proteins, e.g., collagen, laminin, fibronectin, vitronectin, tenascin, integrin, glycosaminoglycan (hyaluronic acid, chondroitin sulfate, dermatan sulfate, heparan sulfate, heparin, keratan sulfate and the like), elastin and fibrin. In some embodiments, growth factors and/or cytokines, such as vascular endothelial cell growth factors, platelet derived growth factors, epidermal growth factors, fibroblast growth factors, hepatocyte growth factors, insulin-like growth factors, and transforming growth factors are exogenously added to the culture.

The cells may be cultured on a surface of glass, ceramic or a surface-treated synthetic polymer. For example, polystyrene that has been subjected to a surface treatment, like γ-ray irradiation or silicon coating, may be used as a surface for cell culture.

Cells which grow to over 85% confluence form cell sheet layer that may be separated from the surface either mechanically, or by using proteolysis enzymes, such as trypsin or dispase. Non-enzymatic cell dissociation could also be used. A non-limiting example includes a mixture of chelators sold under the tradename CELLSTRIPPER (Mediatech, Inc., Herndon, Va.), a non-enzymatic cell dissociation solution designed to gently dislodge adherent cells in culture while reducing the risk of damage associated with enzymatic treatments.

In another embodiment, cells are cultured on a non-adherent surface at sufficient densities. This provides a cell sheet layer that has only a few structural defects as they are recovered with intracellular desmosome structures and the cell-to-cell connectivity and orientation is being kept intact.

In another embodiment, cells are cultured on thermoresponsive dishes supplied for example, by CellSeed, Inc. (Tokyo, Japan). In this embodiment, the culture surface can be inherently non-adherent or can be rendered non-adherent by surface coatings well known to those skilled in the art. Commercially available cell growth support devices include, for example, the range of Corning® Ultra Low Attachment surface cell culturing products (Corning Inc., Corning N.Y.). These products have a hydrogel layer that is hydrophilic and neutrally charged covalently bound to polystyrene surfaces. Since proteins and other biomolecules passively adsorb to polystyrene surfaces through either hydrophobic or ionic interactions, this hydrogel surface naturally inhibits nonspecific immobilization via these forces, thus inhibiting subsequent cell attachment. Other biocompatible non-adherent materials include ePTFE, polystyrene, stainless steel, and some cross-linked cellulose derivatives. Examples thereof include cross-linked hydroxyalkyl celluloses e.g. hydroxyethyl cellulose, hydroxypropyl cellulose, methyl, thyl and methyl thyl celluloses. Cross-linked carboxyalkyl celluloses also included are carboxymethyl cellulose cross-linked with ethylene glycol diglycidyl ether (EGDGE) or 1,4 butanediol diglycidyl ether. Other materials include polyvinyl alcohol, poly(2-hydroxyethyl methacrylate) (Cellform® (MP Biomedicals, Irvine, Calif.), agarose, and crosslinked agarose.

Cells can also be seeded into or onto a natural or synthetic three-dimensional support matrix such as a preformed collagen gel or a synthetic biopolymeric material. Use of attachment factors or a support matrix with the medium of the present invention will enhance cultivation of many attachment-dependent cells in the absence of serum supplementation.

The cell seeding densities for each experimental condition can be selected for the specific culture conditions being used. For routine culture in plastic culture vessels, an initial seeding density of, for example, 1−5×10⁴ cells per cm² is useful. In certain cases, micromass cultures are used.

Cells may be genetically altered by the introduction of a heterologous nucleic acid (e.g. DNA), using various methods known in the art including calcium-phosphate- or DEAE-dextran-mediated transfection, protoplast fusion, electroporation, liposome mediated transfection, direct microinjection and adenoviral or retroviral infection.

In a specific embodiment, a calcium-phosphate precipitate containing DNA encoding the gene(s) of interest can be prepared using the technique of Wigler et al. (Proc. Natl. Acad. Sci. USA 76:1373-1376, 1979). Cultures of adult stem cells (e.g., liver stem cells or adipose stem cells) or their progeny are established in tissue culture dishes. Twenty-four hours after plating the cells, the calcium phosphate precipitate containing approximately 20μg/ml of the heterologous DNA is added. The cells are incubated at room temperature for 20 minutes. Tissue culture medium containing 3 μM chloroquine is added and the cells are incubated overnight at 37° C. Following transfection, the cells are analyzed for the uptake and expression of the foreign DNA. The cells may be subjected to selection conditions that select for cells that have taken up and expressed a selectable marker gene.

Selectable marker genes include, but are not limited to, GFP (green fluorescence protein) or a drug resistance gene. Some non-limiting examples of drug-resistance genes for use in the invention include hygromycin resistance gene, neomycin resistant gene, ampicillin resistance gene, E. coli gpt gene or the like.

In another specific embodiment, the heterologous DNA is introduced into a multipotent stem cell using the technique of retroviral transfection. Various processes are known in the art for transferring retroviral vectors into cultured cells. For example, recombinant retroviruses harboring the gene(s) of interest are produced in packaging cell lines to produce culture supernatants having a high titer of virus particles (for example, 10⁵-10⁶ pfu/ml). The recombinant viral particles are used to infect cultures of the stem cells (e.g., adult liver stem cells or adult adipose stem cells) or their progeny by, for example, incubating the cell cultures with medium containing the viral particles and 8 μg/ml polybrene for three hours. Following retroviral infection, the cells are rinsed and cultured in standard medium. The infected cells are then analyzed for the uptake and expression of the heterologous DNA. The cells can be subjected to selective conditions that select for cells that have taken up and expressed a selectable marker gene. Since the gene transferred by the retroviral vector is integrated into chromosomal DNA of the host stem cell, the gene is transmitted to the daughter cell and therefore can be expressed stably over long period.

The stem or stem-like cells may be subjected to conditions to produce a differentiated cell. The differentiated cell may be of any kind, and a skilled artisan recognizes how to tailor differentiation conditions to result in the desired differentiated cell. In specific cases, the differentiated cell is a neuron, blood cell, muscle cell, or skin cell, for example. Differentiation conditions include culturing cells ex vivo in media containing growth factors that induce differentiation into neuron, blood cell, muscle cell, or skin cell, for example. In certain embodiments, the stem-like cells are differentiated into specific cell types, such as epithelial cells, stromal cells, cardiac cells, bone cells and more. As is readily apparent to those skilled in the art, there are several methods known and under current development for the differentiation of stem/progenitor cell lines into differentiated target cell types. The present disclosure is not to be limited by the specific methods used to induce differentiation, but rather includes use of all such methods that are operationally defined as yielding the desired differentiation into a fully differentiated cell type. For example, U.S. Pat. Nos. 6,596,274, and 5,811,094 disclose methods for cell differentiation.

Exemplary differentiated cells include but are not limited to fibroblasts, keratinocytes, epithelial cells, endothelial cells, neural cells, epidermal cells, hematopoietic cells, melanocytes, chondrocytes, hepatocytes, B-cells, T-cells, erythrocytes, macrophages, monocytes, muscle cells, vascular smooth muscle cells, stem cells, differentiated cells, plant cells, mammalian cells, mesenchymal cells, oral and gastrointestinal mucosal epithelia cells, urinary tract epithelia cells, vascular endothelial cells, neural cells, epidermal cells, osteoblasts, intervertebral disc cells, pancreatic cells, angioblasts, bone marrow, mesenchymal cells, myoblasts, cardiomyocytes, amniotic cells, and placental cells.

C. HSC Characterization

In some embodiments of the method, the stem or stem-like cells are analyzed for expression of one or more genes, including one or more genes whose expression is indicative of an induced pluripotent stem cell phenotype. In some aspects, the one or more pluripotent stem cell markers include one or more of OCT4, SOX2, UTF1, REX1, OXT2, NANOG, UTF1 AC133, CD9, DNMT3B, FOXD3, ALP, TERT, TERF, FZD9, GCNF, and SCGF. In specific aspects, the cells are assayed for expression of one or more genes selected from the group consisting of OCT4, SOX2, KLF4, and NANOG.

The process of making a differentiated cell from a stem or stem-like cell is accompanied by changes in the expression of cell markers. There are also unique pluripotent stem cells markers as well as markers of multilineage differentiation. Such cell markers are typically expressed as mRNA and/or protein. Detection of the mRNA or protein markers may be performed by any method known in the art. In some embodiments, nucleic acids and/or proteins will be isolated from the cells and then analyzed.

Protein markers can be detected using any suitable immunological technique such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium.

The expression of pluripotent or tissue-specific markers can also be detected at the mRNA level by Northern blot analysis, dot-blot hybridization analysis, or by reverse transcriptase-initiated polymerase chain reaction (RT-PCR) using sequence-specific primers in standard amplification methods. See for example, U.S. Pat. No. 5,843,780. Sequence data for the particular markers can be obtained from public databases such as GenBank. Expression of tissue-specific markers as detected at the protein or mRNA level is considered positive if the level is at least 2-fold, and, in certain instances, more than 10-, 50-, 100-, 150- or 200-fold above that of a control cell, such as an undifferentiated adult liver stem cell, a fibroblast, or other unrelated cell type.

D. Engineering of HSCs

The genetic engineering of hematopoietic stem cells is the basis for potentially treating a large array of hereditary and acquired diseases. HSCs are harvested from the subject, genetically engineered ex vivo and re-transplanted or infused into the same subject after administration of a conditioning treatment that favors their engraftment in the bone marrow.

Conditioning is comprised of chemotherapy, radiation therapy, or both. It is applied to kill any remaining cancer cells in the recipient's body, make room for the donor stem cells in their marrow spaces, and suppress the immune system so that the recipient can accept the donor stem cells.

The engineered and engrafted HSCs provide a steady supply of genetically engineered progeny potentially for the recipient's lifetime. Mature cells of myeloid and lymphoid lineages may then reverse pathological conditions such as inherited immune deficiencies, blood and storage disorders, infections, and cancer. Myeloid lineage includes monocytes, macrophages, neutrophils, basophils, eosinophils, erythrocytes, and megakaryocytes to platelets. Lymphoid cells include T cells, B cells, and natural killer cells.

Genetic engineering of HSCs requires inserting the therapeutic gene into the cellular chromosomal DNA, or editing a selected nucleotide sequence, to ensure maintenance of gene correction as the cell replicate its genome and transmit it to the progeny, whether during self-renewal or in the output of differentiating cell lineages. The most commonly used gene transfer tools rely on the ability of retroviruses and lentiviruses to integrate into the chromatin of infected cells and are obtained by engineering the virus into replication-defective vehicles (vectors/plasmids) of self-contained gene expression cassettes.

Vectors provided herein are designed to permit expression of TRAIL and DcR1 under the control of regulated eukaryotic promoters (i.e., constitutive, inducible, repressable, tissue-specific). One of skill in the art would be well-equipped to construct a vector through standard recombinant techniques. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), and lentiviral vectors (e.g. derived from HIV-1, HIV-2, SIV, BIV, FIV etc.).

Viral vectors utilize viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

More broadly, retroviruses (including lentiviruses) are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types; however, integration and stable expression require the division of host cells.

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination.

### V. METHODS OF USE

Certain aspects of the present disclosure, the engineered HSCs can be used to prevent or treat a disease or disorder such as cancer. Provided herein, in certain embodiments, are methods for treating or delaying progression of cancer in an individual comprising administering to the individual an effective amount an engineered HSC therapy. Examples of cancers contemplated for treatment include lung cancer, head and neck cancer, breast cancer (including triple-negative breast cancer), pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, the cancer may be resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

A. Pharmaceutical Compositions

Where clinical application of a therapeutic composition containing an inhibitory antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

Also, provided herein are pharmaceutical compositions and formulations comprising engineered HSCs and a pharmaceutically acceptable carrier. The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The active compounds can be formulated for parenteral administration, e.g., formulated for injection via intratumoral, intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

B. Additional Therapy

In certain embodiments, the compositions and methods of the present disclosure may employ a second or additional cancer therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an engineered HSC theapy and a second therapy. A tissue, tumor, or cell can be contacted with one or more compositions or pharmacological formulation(s) comprising one or more of the agents, or by contacting the tissue, tumor, and/or cell with two or more distinct compositions or formulations.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An engineered HSC cell therapy as described herein may be administered before, during, after, or in various combinations relative to an anti-cancer treatment. The administrations may be in intervals ranging from concurrently to minutes to days to weeks. In embodiments where the engineered HSC cell therapy as described herein is provided to a patient separately from another anti-cancer agent, one would generally ensure that a significant period of time did not expire between the time of each delivery, such that the two therapies would still be able to exert an advantageously combined effect on the patient. In such instances, it is contemplated that one may provide a patient with the engineered HSC cell therapy as described herein and the other anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, the additional therapy may also be the administration of side-effect limiting agents (e.g., agents intended to lessen the occurrence and/or severity of side effects of treatment, such as anti-nausea agents, etc.).

Various combinations may be employed. For the example below an engineered HSC cell therapy as described herein is “A” and the other anti-cancer therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody—drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art such as described in U.S. Patent Publication Nos. US20140294898, US2014022021, and US20110008369, all of which are incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

C. Kits

In various aspects of the embodiments, a kit is envisioned containing therapeutic agents and/or other therapeutic and delivery agents. In some embodiments, the present disclosure contemplates a kit for preparing and/or administering a therapy of the embodiments. The kit may comprise one or more sealed vials containing any of the pharmaceutical compositions of the present embodiments. The kit may include, for example, engineered HSC cell as described herein as well as reagents to prepare, formulate, and/or administer the engineered HSC cells as described herein. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent).

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

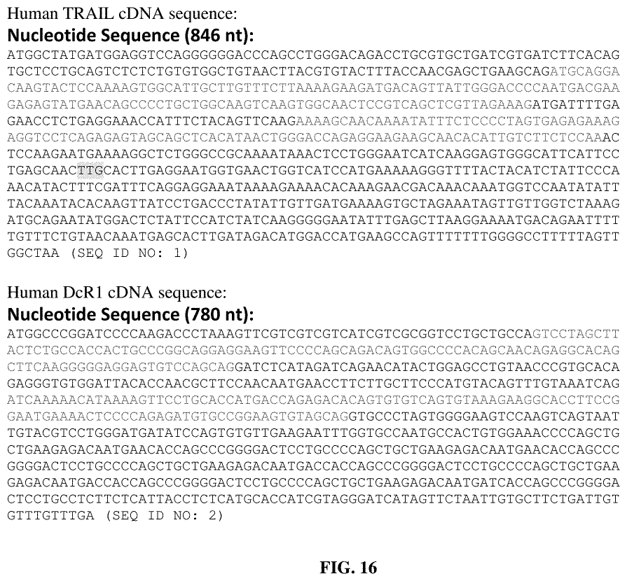

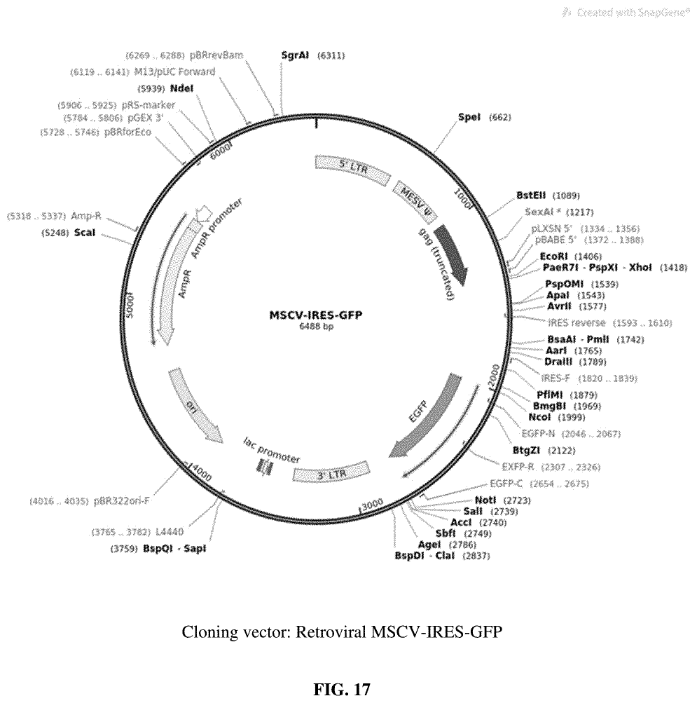

Sequences. Human TRAIL and human DcR1 cDNA has been cloned in the EcoRI site of the MSCV-IRES-GFP vector (see FIGS. 16-17).

Retroviral vectors and vector production. The coding region of human TRAIL and human DcR1 was amplified by polymerase chain reaction (PCR) and sub-cloned into EcoRI sites in the MSCV-IRES-GFP vector (ADDGENE). Helper-free recombinant retrovirus produced in supernatants of transfected ecotropic Phoenix packaging cell line was used to transduce the ecotropic GP+E86 packaging cell line. Helper-free recombinant retrovirus produced in supernatants of the transfected ecotropic Phoenix packaging cell line was used to transduce the ecotropic GP+E86 packaging cell line. GP+E86-hu TRAIL and GP+E86-hu DcR1 producer cell lines were maintained and expanded for virus production and collection.

Mice and retroviral infection of primary bone marrow cells. 6-8 weeks old female BALB/c mice were purchased from Charles River, kept in pathogen free conditions at the animal laboratory of Vanderbilt University. All animal experiments were approved by the IACUC. Primary mouse bone marrow cells were harvested from mice treated with 150 mg of 5-fluorouracil/kg 4 days before harvest and stimulated for 48 hours in Dulbecco's modified Eagle's medium (DMEM) (STEM cell Technologies, Inc.) supplemented with 15% fetal bovine serum, 10 ng/mL of human interleukin-6 (hIL-6), 6 ng/mL of murine interleukin-3 (mIL-3), and 20 ng/mL of murine stem cell factor (mSCF). The cells were transduced by co-spinoculation twice at a time interval of 12 hours with concentrated huTRAIL and huDcR1 retroviral supernatant in the presence of 5 μg/mL protamine sulfate at 2000 rpm, 30° C. for 60 minutes. Cells were then maintained in 6 ng/ml mIL-3, 10 ng/ml hIL-6, and 20 ng/ml mSCF for 36 hours following flow cytometric analysis for GFP and apoptosis, and transplantation.

Bone marrow transplantation and monitoring of mice. Retrovirally transduced bone marrow cells were injected into the tail vein of semi-lethally irradiated syngeneic recipient mice that were exposed to a single dose of 650 cGy. Donor chimerism was monitored by tail vein bleeds and FACS analysis of GFP expressing cells every two weeks.

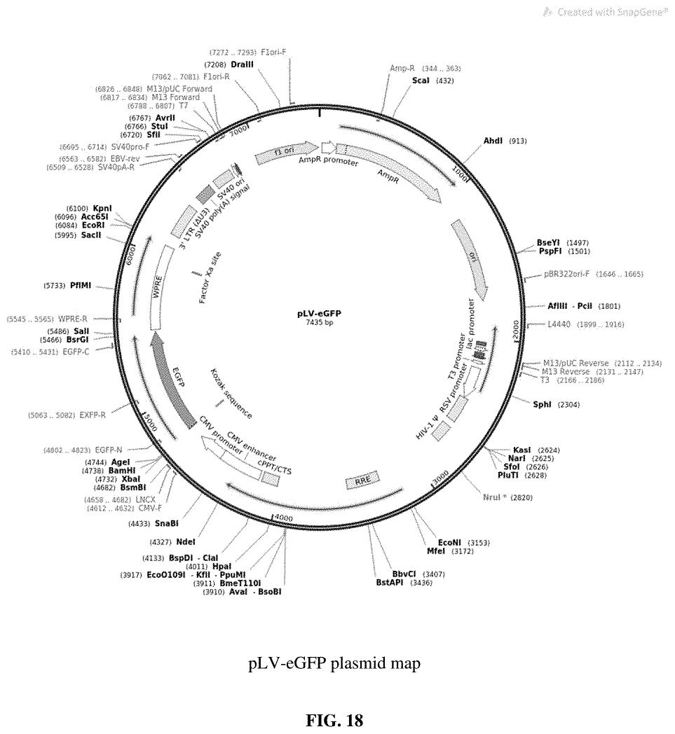

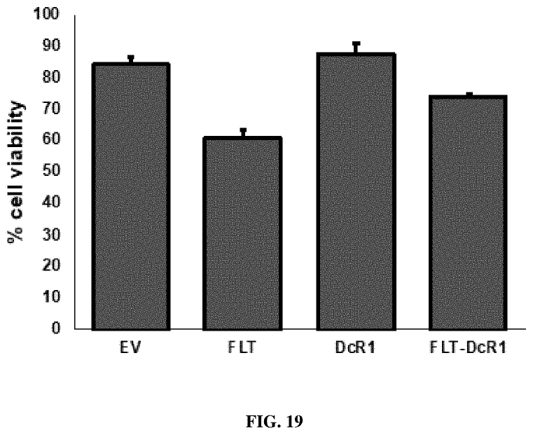

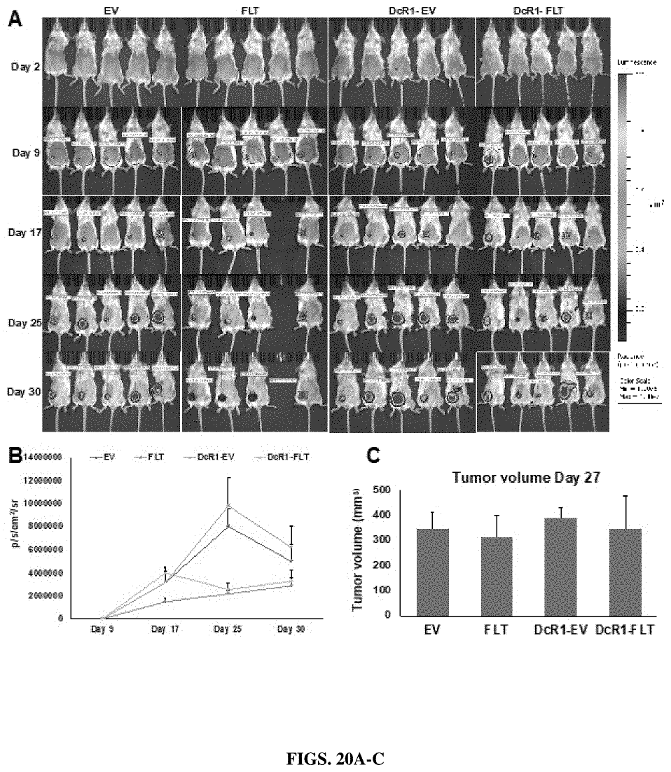

A new lentiviral plasmid has been engineered to co-expresses TRAIL and DcR1 on a single plasmid. The plasmid map of pLV-eGFP (ADDGENE) is shown in FIG. 18.

The cDNAs of huTRAIL and huDcR1 are sub-cloned in the multiple cloning site of this plasmid. The advantage of this new plasmid is that a single plasmid is used for producing both TRAIL and DcR1 in transduced cells. This avoids the use of two different plasmids during transfection and associated toxicity in the cells.

### Example 2—Results

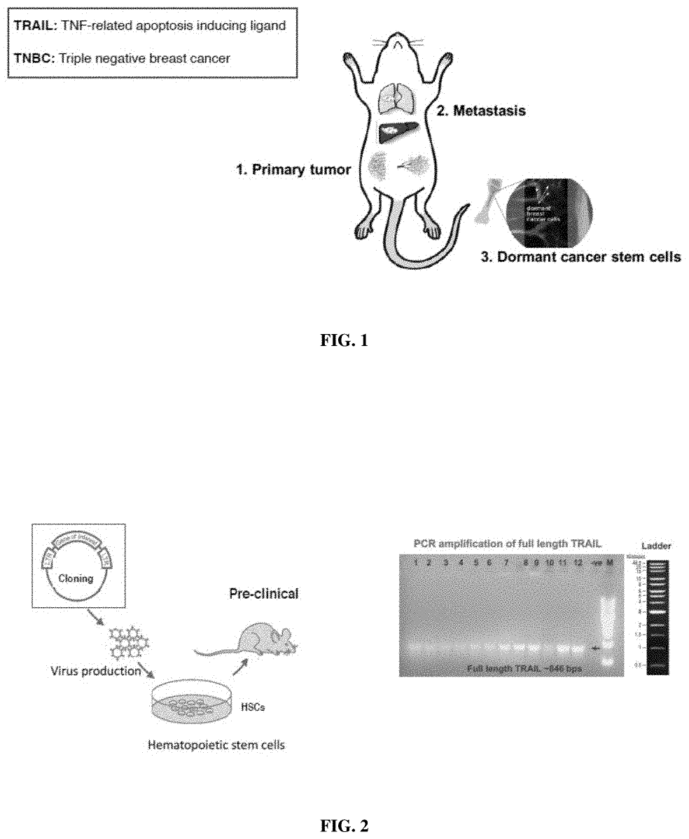

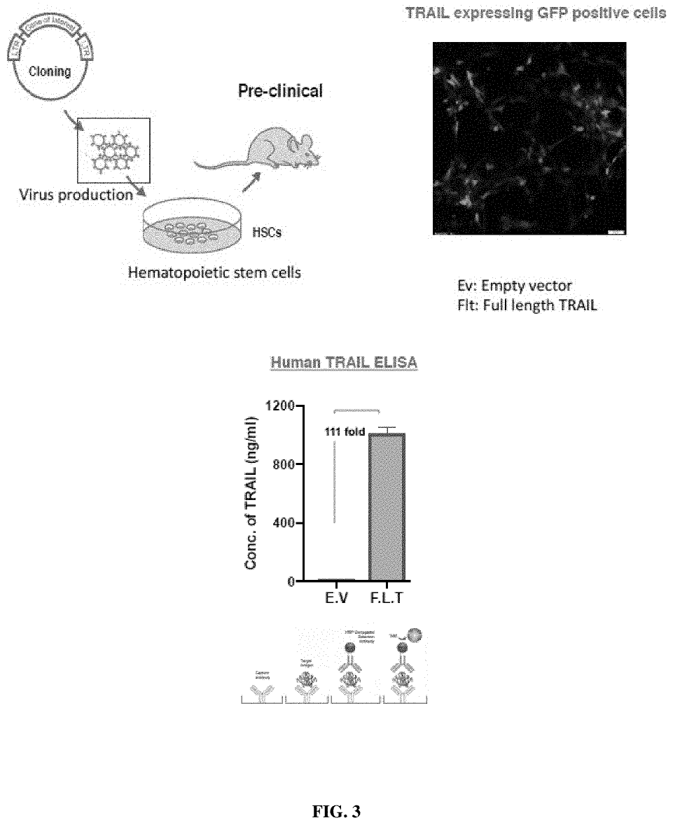

Following the hematopoietic stem cell genetic manipulation using the transplantation schematics described (FIGS. 17-18), full length human Trail in a GFP expressing retroviral MSCV vector was first sequenced. This vector was used to transduce and sort the virus producer cell line that stably expresses trail retrovirus as can be seen that all these cells express GFP. The TRAIL expression was confirmed by performing TRAIL ELISA. A110-fold increase was increased in the levels of TRAIL in the supernatant of these cells (FIGS. 2-3).

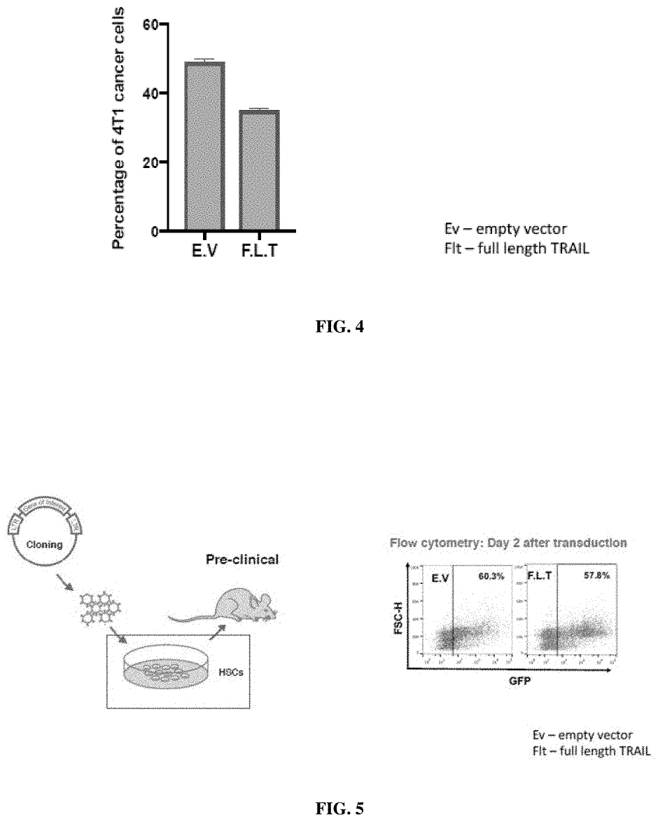

When the TRAIL producer cell line was co-cultured with 4T1 breast cancer cells in a 50:50 ratio, a decrease in the percentage of 4T1s was observed when cultured with TRAIL producer cells compared to empty vector (FIG. 4). The next step was to transduce the hematopoietic stem cells for characterization and in vivo transplantation. Bone marrow cells were isolated from a healthy donor mouse and these cells depleted for lineage to obtain the stem and progenitor population. The retroviral transduction of HSCs results in 60 to 75% of GFP positive HSCs in a reproducible manner (FIG. 5). Engineered HSCs were further characterized for TRAIL production and found a 25-fold increase in TRAIL in the supernatant of these cells (FIG. 6).

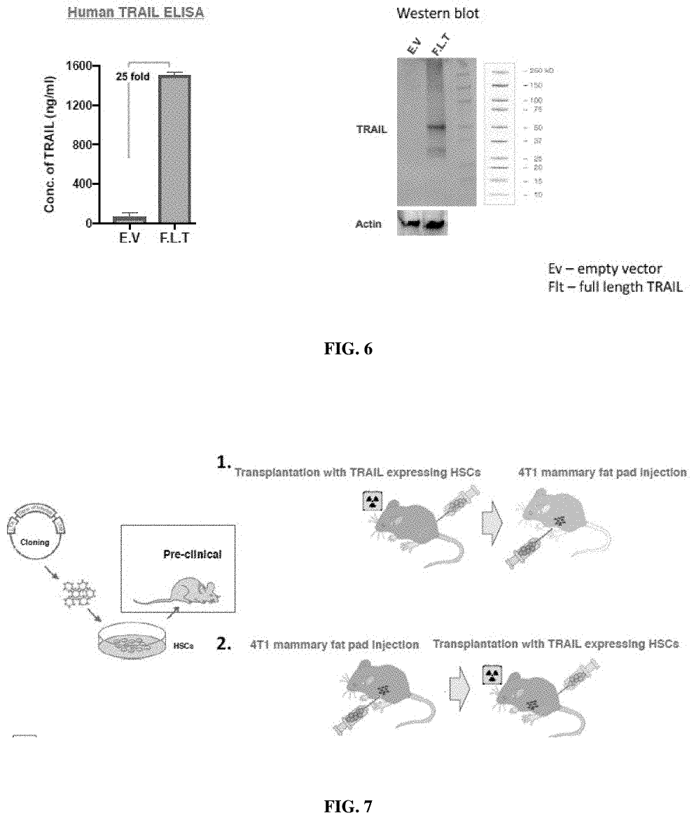

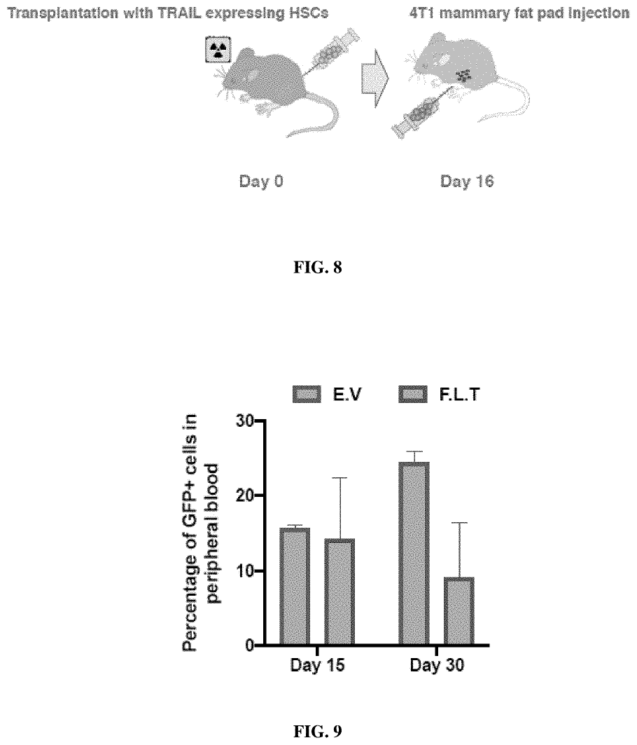

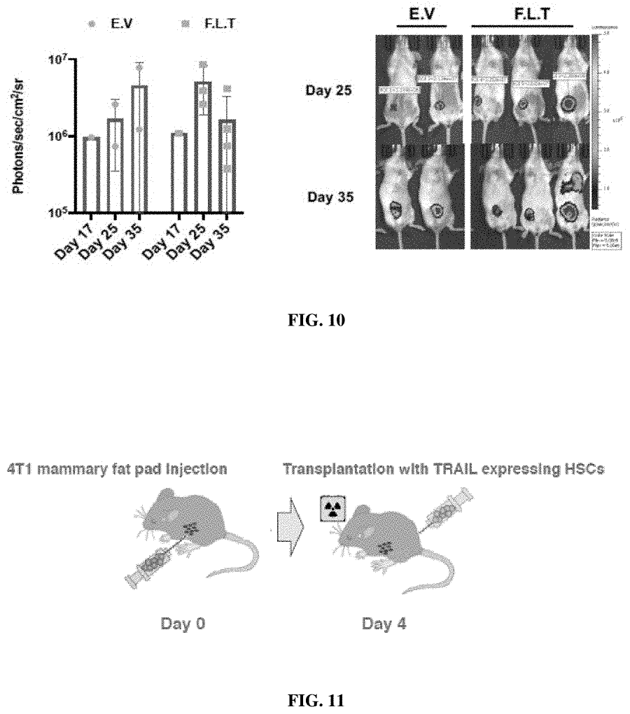

For the next step of testing in mice there were two choices: (a) transplantation of engineered hematopoietic stem cells and then inoculation of 4T1 tumor cells, or (b) inoculation of 4T1 tumor cells followed by HSC transplantation (FIG. 7). The transplantation was performed first and lethally irradiated and transplanted the recipient mice with engineered HSCs t that produce TRAIL and GFP followed by 4T1 injections (FIG. 8). Blood was drawn from these mice via tail vein bleeding at different time points to monitor the engraftment of donor cells in the peripheral blood. The bleeding analysis of these mice suggested almost similar engraftment of empty vector and TRAIL HSCs at two weeks, however the % of trail expressing cells went down with time (FIG. 9). 4T1 TNBC cells were injected in these mice at day 16, and the engraftment of donor cells confirmed at day 15 in the previous slide. and imaged for luciferase signal of cancer cells at day 16. A decrease was observed in luciferase signal in the TRAIL group from day 25 to day 35. Though a decrease was observed in the signal and a visible necrotic core at primary tumor site, this was only a transient therapeutic effect, mostly because of a decrease in the percentage of TRAIL expressing cells (FIG. 10).

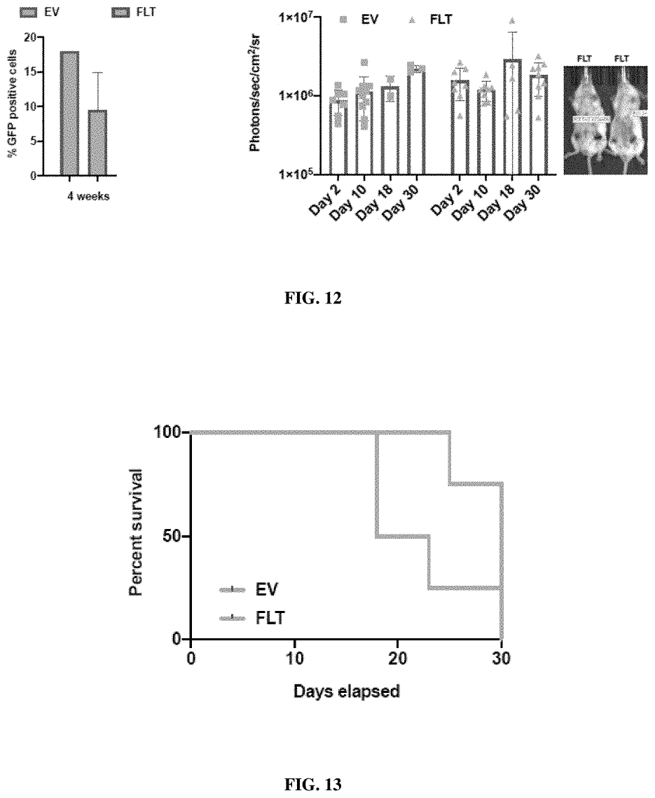

In a second in vivo experiment, 4T1 tumor cells were first injected on both sides of the fourth mammary fat pad followed by HSC transplantation (FIG. 11). FIG. 12 shows the percentage of GFP positive cells in peripheral blood of the transplanted mice and again, a proliferative disadvantage of TRAIL expressing HSCs is observed. Again, a therapeutic benefit is observed between day 2 and day 10 and between day 18 and day 30. In these mice, a visible necrotic core was observed in the tumor. This effect was transient as these mice eventually died or had to be sacrificed because of tumor size and metastatic burden. A trend of increased survival in TRAIL -HSCs transplanted group was observed (FIG. 13).

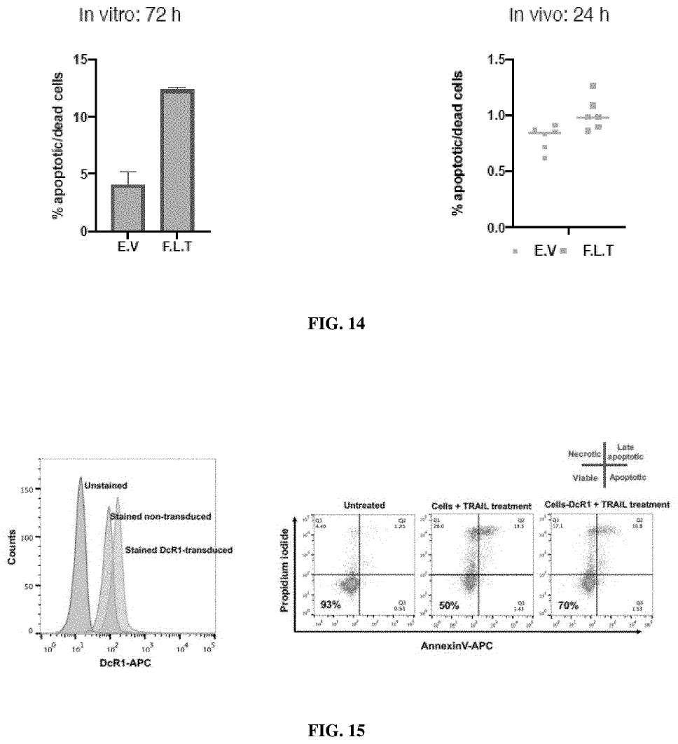

In all in vivo experiments, it was clearly observed that TRAIL expressing HSCs do not have proliferative advantage in vivo and that they decrease in number with time (FIG. 14). It was hypothesized that the overexpression of TRAIL in these cells may lead to their apoptosis. So, to confirm this, the percentage of apoptotic cells in TRAIL expressing HSCs both in vitro and in vivo was quantified and increased apoptosis observed in HSCs that overexpress TRAIL. FIG. 15 shows that the K562 leukemia cells overexpress DcR1 following transduction with DcR1-retroviral vector and as expected this can rescue them from TRAIL-mediated apoptosis significantly.

Equal number of lineage negative mouse hematopoietic stem and progenitor cells isolated from a normal healthy mouse, were transduced with empty GFP vector (EV), full length TRAIL (FLT), Decoy receptor 1(DcR1) and full-length TRAIL plus DcR1 (FLT-DcR1), at an MOI of 10. For the DcR1 and FLT-DcR1, the inventors combined MOI 5 from each FLT and DcR1 and, EV. Following transduction, the cells were kept in the incubator for 72 h. The viability of transduced cells was analyzed via annexin V apoptosis assay. The inventors observed a slight increase in the viability of cells that were transduced with FLT-TRAIL compared to alone FLT transduced cells.

In a 3^{rd} in vivo experiment, at day0 100,000 GFP+ve cells (lineage negative mouse hematopoietic stem and progenitor cells isolated from a normal healthy mouse, transduced with empty GFP vector (EV), full length TRAIL (FLT), Decoy receptor 1 plus empty vector (DcR1) and, full-length TRAIL plus DcR1 (FLT-DcR1), at an MOI of 10) were transplanted in lethally irradiated healthy Balb/c mice. Mice were injected with 30,000 4T1-luc cells in their 4^{th} mammary fat pad at day 1. These mice were regularly monitored (day 2, 9, 17, 25, 30) for the luciferase signal from 4T1-luc cells and development of primary tumor, via IVIS imaging. Quantitation of IVIS imaging signal in these mice suggested a slow tumor growth in FLT mice. A significant decrease in the luciferase signal was observed from day 17 to day 25 in FLT-DcR1 mice compared to other groups. Overall, FLT-DcR1 and FLT mice showed a reduced or slower increase in the primary tumor signal suggesting an anti-tumor effect of TRAIL in vivo. Notably, we did not see any significant difference in the tumor volume among all the groups. The inventors believe that using two different expression plasmids for transduction of HSCs has some negative effects and probably this is the reason that we did not see a drastic effect of FLT and DcR1 co-expression in vivo. Further studies utilizing a single lentiviral construct that expresses both, FLT and DcR1 are needed to see a better therapeutic effect of transduced hematopoietic stem cell transplantation.

In summary, while HSCs can be genetically engineered to produce therapeutic TRAIL in preclinical settings the TRAIL expression decreases with time in vivo. The inventors here have determined that decoy receptor co-expression can inhibit TRAIL-mediated apoptosis in cells, thereby extending the in vivo efficacy of HSC TRAIL therapies.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An engineered cell that expresses Decoy Receptor 1 (DcR1) and TNF-Related Apoptosis-Inducing Ligand (TRAIL) and exhibits reduced activation of caspase-dependent apoptosis as compared a cell engineered to express TRAIL but not engineered to express DcR1.

2. The engineered cell of claim 1, wherein said engineered cell overexpresses TRAIL as compared to a non-engineered cell.

3. The engineered cell of claim 1, wherein said engineered cell overexpresses DcR1 as compared to a non-engineered cell.

4. The engineered cell of claim 1, wherein said engineered cell overexpresses TRAIL and DcR1 as compared to a non-engineered cell.

5. The engineered cell of claims 1-4, wherein said engineered cell is a stem cell.

6. The engineered cell of claims 1-4, wherein said engineered cell is a hematopoietic stem cell (HSC).

7. The engineered cell of claims 1-6, wherein coding regions for DcR1 and TRAIL are under control of the same promoter.

8. The engineered cell of claim 7, wherein coding regions for DcR1 and TRAIL are separated by an internal ribosome entry site

9. The engineered cell of claim 7, wherein DcR1 or TRAIL are encoded as a polyprotein having a protease cleavage site disposed between DcR1 and TRAIL sequences.

10. The engineered cell of claims 1-6, wherein coding regions for DcR1 and TRAIL are under control of different promoters.

11. The engineered cell of claims 1-10, wherein the engineered cell is a non-human mammalian cell, such as a murine cell.

12. The engineered cell of claims 1-10, wherein the engineered cell is a human cell.

13. The engineered cell of claims 1-12, wherein the engineered cell further expresses a detectable marker protein.

14. The engineered cell of claim 1-13, wherein the engineered cell further encodes an inducible suicide gene.

15. A method of killing a cancer cell comprising contacting said cancer cell with an engineered cell of claims 1-14.

16. The method of claim 15, wherein the cancer cell is a breast cancer cell, a lung cancer cell, a skin cancer cell, a brain cancer cell, a head & neck, a lymphatic cancer cell, a pancreatic cancer cell, a liver cancer cell, a bladder cancer cell, a stomach cancer cell, a colon cancer cell, a prostate cancer cell, a uterine cancer cell, a cervical cancer cells, a testicular cancer cell, a lymphoma cell, a leukemia cell.

17. The method of claim 15, wherein the cancer cell is a triple negative breast cancer cell.

18. The method of claims 15-17, wherein the cancer cell is a recurrent, metastatic or drug resistant cancer cell.

19. The method of claims 15-18, wherein contacting comprises intratumoral administration, administration into the tumor vasculature, or administration regional to the cancer.

20. The method of claims 15-19, wherein contacting comprises systemic administration.

21. The method of claim 20, wherein systemic administration comprises intravascular (intravenous, intra-arterial), intraperitoneal, subcutaneous or topical administration.

22. The method of claims 15-21, further comprising contacting said cancer cell with a second cancer therapy, such as chemotherapy, radiotherapy, immunotherapy, hormonal therapy, toxin therapy.

23. The method of claims, 15-22, wherein said cancer cell is located in a living subject, and said method further comprises a conditioning treatment to improve engraftment of said engineered cell.

24. The method of claim 23, further comprising performing surgery on said living subject to resect said cancer cell.

25. The method of claims 23-24, wherein said living subject is a human or non-human mammal.
